(19) United States
(12) Patent Application Publication
(10) Pub. No.: US 2012/0201886 A1
(43) Pub. Date: Aug. 9, 2012

(54) Coated Extended Release Pharmaceutical Compositions Containing Paliperidone

(75) Inventors: Rajesh Kshirsagar, Bangalore (IN); Ganesh SHINDE, Bangalore (IN); Pravin KAMBLE, Bangalore (IN)

(73) Assignee: MICRO LABS LIMITED, Bangalore (IN)

(21) Appl. No.: 13/194,353

(22) Filed: Jul. 29, 2011

(30) Foreign Application Priority Data
Jul. 30, 2010 (IN) 2182/CHE/2010

(51) Int. Cl. A61K 31/519; A61K 9/00; A61K 9/22

(52) U.S. Cl. 424/468; 424/400; 514/259.41

(57) ABSTRACT

A non-osmotic coated extended release pharmaceutical composition comprising Paliperidone or pharmaceutically acceptable salts thereof and one or more pharmaceutical excipients and process for preparing the same. The present invention particularly relates to a non-osmotic coated extended release pharmaceutical composition comprising Paliperidone or pharmaceutically acceptable salts thereof and one or more pharmaceutical excipients wherein the core is coated with a release controlling composition.

### TECHNICAL FIELD OF THE INVENTION

The present invention relates to a non-osmotic coated extended release pharmaceutical composition comprising Paliperidone or pharmaceutically acceptable salts thereof and one or more pharmaceutical excipients and process for preparing the same.

### BACKGROUND OF THE INVENTION

Paliperidone has the chemical name (RS)-3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one. Paliperidone is practically insoluble in water, freely soluble in methylene chloride and soluble in methanol and 0.1 N hydrochloric acid. Presently Paliperidone is available as INVEGA® Extended-Release Tablets in 1.5 mg, 3 mg, 6 mg and 9 mg strengths. INVEGA® utilizes OROS® osmotic drug-release technology. INVEGA® utilizes osmotic pressure to deliver Paliperidone at a controlled rate. The delivery system consists of an osmotically active trilayer core surrounded by a subcoat and semipermeable membrane. The trilayer core is composed of two drug layers containing the drug and excipients, and a push layer containing osmotically active components. There are two precision laser-drilled orifices on the drug-layer of the tablet. In an aqueous environment, such as the gastrointestinal tract, the water-dispersible overcoat erodes rapidly. Water then enters the tablet through the semipermeable membrane that controls the rate at which water ingress the tablet core, which, in turn, determines the rate of drug delivery. The hydrophilic polymers of the core hydrate and swell creating a gel containing Paliperidone that is then pushed out through the tablet orifices. The biologically inert components of the tablet remain intact during gastrointestinal transit and are eliminated in the stool as a tablet shell, along with insoluble core components.

Many oral osmotic dosage forms of Paliperidone are disclosed in WO 2004010981 A1, WO 2006/085856 A1, WO 2007/044234 A1, WO 2007/050377 A1. WO2006/017537 discloses dosage form which shows ascending rate of release over an extended period of time.

There are various disadvantages associated with osmotic drug-release technology; such as this technology requires highly sophisticated equipments for processes like compression, coating and laser drilling. Further osmotic drug-release technology requires special excipients like osmogen, osmopolymer, polymer for semipermeable membrane, which ultimately increases cost of manufacturing. Also while preparing osmotic dosage forms using laser drilling the drilling may not performed and such faulty dosage form may not able to release active at all.

U.S. patent application publication No. US 2006/034927 discloses a Paliperidone dosage form for sustained release of a drug comprising: a delay layer comprising (i) a polymeric matrix, and (ii) microencapsulated drug, wherein the delay layer is substantially free of non-microencapsulated drug; and a second layer comprising (iii) a polymeric matrix, and (iv) non-microencapsulated drug matrix; wherein the second layer is located adjacent to the delay layer.

Thus there is still unmet need to develop a simple, stable, extended release solid oral pharmaceutical composition of Paliperidone, which does not require highly precise technique like drilling on the dosage form and which can provide compositions which are simple to manufacture, cost effective with stable compositions and acceptable dissolution profile.

### SUMMARY OF THE INVENTION

In one aspect the present invention provides a non-osmotic coated extended release pharmaceutical composition comprising Paliperidone or pharmaceutically acceptable salts thereof and one or more pharmaceutical excipients for once daily dosing.

In yet another aspect the present invention provides a process for preparation of a non-osmotic coated extended release pharmaceutical composition comprising Paliperidone or pharmaceutically acceptable salts thereof and one or more pharmaceutical excipients for once daily dosing.

In yet another aspect the present invention provides a non-osmotic coated extended release pharmaceutical composition comprising Paliperidone or pharmaceutically acceptable salts thereof and one or more pharmaceutical excipients for once daily dosing which is bioavailable and effective with sufficient shelf-life, good pharmaceutical properties, enhancing patient compliance and reducing possible side effects.

In yet another aspect the present invention provides a non-osmotic coated extended release pharmaceutical composition comprising Paliperidone or pharmaceutically acceptable salts and one or more pharmaceutical excipients for once daily dosing, which can be prepared in dosage forms of different strength by proportionally adjusting the quantities of the excipients and the active ingredient, thereby providing a pharmaceutical linearity, without affecting the dissolution (profile and bioavailability of the active ingredient.

### DETAILED DESCRIPTION OF THE INVENTION

The present invention relates a non-osmotic coated extended release pharmaceutical composition wherein Paliperidone is present in immediate release core and core is coated with a release controlling composition to control the release rate of Paliperidone. The controlled release coating solely controls the release of Paliparidone. This design is especially useful in controlling and/or tailoring the desired release of the drug, especially providing relatively low doses of Paliperidone over a prolonged period.

The present invention provides a non-osmotic coated extended release pharmaceutical composition comprising Paliperidone or pharmaceutically acceptable salts thereof and one or more pharmaceutical excipients for once daily dosing wherein the core is coated with a release controlling composition wherein the release of active is solely controlled by coating comprising release controlling composition.

The term “extended release” herein refers to any formulation or dosage form that comprises an active drug and which is formulated to provide a longer duration of pharmacological response after administration of the dosage form than is ordinarily experienced after administration of a corresponding immediate release formulation comprising the same drug in the same amount. Controlled release formulations include, inter alia, those formulations described elsewhere as “controlled release”, “delayed release”, “sustained release”, “prolonged release”, “programmed release”, “time release” and/or “rate controlled” formulations or dosage forms. Further for the purposes of this invention refers to release of an active pharmaceutical agent over a prolonged period of time, such as for example over a period of 8, 12, 16 or 24 hours.

By “pharmaceutically acceptable” is meant a carrier comprised of a material that is not biologically or otherwise undesirable.

The term “Paliperidone” as used in the invention is meant to cover Paliperidone in the form of freebase or its pharmaceutically acceptable salt(s), hydrate(s), solvate(s) and physiologically functional derivative(s) and precursors thereof. The term also includes all polymorphic forms, whether crystalline or amorphous.

The term ‘non-osmotic’ as used in the invention is meant to cover compositions which do not have ‘osmotically active component i.e. osmogen’, ‘precision laser-drilled orifices’ to release active and ‘semipermeable membrane’ that controls the release rate of active.

In a preferred embodiment, the pharmaceutical composition of the present invention comprises 0.1-50% w/w of Paliperidone or pharmaceutically acceptable salts thereof; preferably the present invention comprises 0.1-25% w/w of Paliperidone or pharmaceutically acceptable salts thereof.

The pharmaceutical compositions of the present invention can be any solid dosage form for example, but not limited to, granules, pellets and tablets. The core dosage forms can be prepared by any of the means using excipients well known to the person skilled in the art.

In a preferred embodiment, the coated extended release pharmaceutical composition comprising Paliperidone or pharmaceutically acceptable salts thereof and one or more pharmaceutical excipients for once daily is in the form of a tablet. The core of the coated extended release tablet composition comprises Paliperidone or pharmaceutically acceptable salts thereof and one or more pharmaceutical excipients

The pharmaceutical compositions according to present invention will, in general comprise of one or more excipients. Examples of pharmaceutical excipients include, but are not limited to binders, fillers or diluents, lubricants, glidants, disintegrants, antioxidants. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The formulation according to present invention may also comprise a disintegrant which may be included in all or part of the oral dosage form to ensure rapid disintegration of the dosage form or part of the dosage form (for example, one of the layers in a bilayer tablet) after administration.

Disintegrants include, but are not limited to: alginic acid, carboxymethylcellulose calcium, carboxymethylcellulose sodium, croscarmellose sodium, crospovidone, guar gum, magnesium aluminum silicate, sodium alginate, sodium starch glycolate and starches and other materials known to one of ordinary skill in the art and combinations thereof.

Antioxidant include, but are not limited to Ascorbic acid, ascorbic palmitate, Vitamin E, butylated hydroxyanisole, butylated hydroxy toluene, hypophosphorous acid, monothioglycerol, propyl gallate, and the like. Preferably antioxidant in the core, ranges from 0.01-2% w/w of the composition.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. One or more of these additives can be selected and used by the skilled artisan having regard to the particular desired properties of the dosage form by routine experimentation without any undue burden.

The amount of each type of additive employed may vary within ranges conventional in the art.

In a preferred embodiment, the core of the present invention is formulated with Paliperidone or pharmaceutically acceptable salts thereof, a diluent, a binder and a lubricant, optional antioxidant. In a more preferred embodiment, the core of the present invention is formulated with Paliperidone or pharmaceutically acceptable salts thereof, lactose monohydrate as diluent, povidone as the binder and magnesium stearate as the lubricant.

The core tablets comprising Paliperidone or pharmaceutically acceptable salts thereof can be prepared by processes well known to those of skill in the art. For example, core tablets can be prepared by wet granulation, dry granulation, melt granulation and the like. In a preferred embodiment, the core tablets comprising Paliperidone or pharmaceutically acceptable salts thereof are prepared by wet granulation.

In a further embodiment, the core tablets are prepared by melt granulation. The core dosage forms comprising Paliperidone or pharmaceutically acceptable salts thereof are then coated with a suitable release controlling composition to control the release rate of Paliperidone or pharmaceutically acceptable salts thereof. The release controlling composition can comprise one or more hydrophilic agents and one or more hydrophobic agents.

Suitable hydrophobic agents include, but are not limited to polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl triethacrylate), poly(isobutyl methacrylate), and poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), Poly(methyl acrylate-co-methyl methacrylate-co-methacrylic acid), Poly(methacrylic acid-co-ethyl acrylate), Poly(methacrylic acid-co-methyl methacrylate), the commercially available Eudragit FS 30D, Eudragit L 100-55, Eudragit L 30D-55, EUDRAGIT® L 100, EUDRAGIT® L 12,5, EUDRAGIT® S 100, EUDRAGIT® S 12,5 Acryl Eze, methy waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, and hydrogenated vegetable oils and the like.

Suitable hydrophilic agents include, but are not limited to water soluble polymers such as hydroxyethyl cellulose, hydroxypropyl cellulose, carboxymethyl cellulose, hydroxypropylmethyl cellulose, sodium carboxymethyl cellulose, vinylpyrrolidone/vinyl acetate copolymer for example marketed as Plasdone® S-630, polyvinyl alcohol, polyethylene glycol and the like. Saccharides such as monosaccharides, disaccharides, oligosaccharides, polysaccharides or sugar alcohols which include but are not limited to sucrose, xylitol, mannitol, sorbitol, glucose, fructose, galactose, maltitol, lactose, maltodextrin. Water soluble organic acids, water soluble salts of organic acids, water soluble organic bases, water soluble salts of organic bases which include but are not limited to citric acid or salts thereof, aminoacids or salt thereof, inorganic salts such as sodium carbonate, sodium bicarbonate, potassium chloride and sodium chloride and the like.

in a preferred embodiment, the coating comprises of a combination of a hydrophobic agent and a hydrophilic agent. The ratio of the hydrophobic agent to the hydrophilic agent is from about 0.1:10 to about 10:0.1.

In a still preferred embodiment of the present invention, the coating comprises from about 2 to 50% w/w of the core, more preferably the coating comprises from about 5 to 40% w/w of the core.

The coating composition may optionally contain other excipients which include, but are not limited to plasticizers, opacifiers, coloring agents and antifoaming agents. Examples of plasticizers include, but are not limited to citrates such as triethyl citrate, acetyl tributyl citrate, phthalates, dibutyl sebacate, triacetin, polyethylene glycol and the like.

Examples of opacifying agents and coloring agents include, but are not limited to titanium dioxide, talc, aluminum lake dyes, insoluble pigments, water-soluble dyes and the like. Antifoaming agents include, but are not limited to silicone, simethicone and the like.

The core tablets can be coated using any of the techniques well known to the persons skilled in the art. In a preferred embodiment, coating of core tablets of Paliperidone is carried out by spraying aqueous and/or non-aqueous solution/dispersion and its mixtures of the coating composition excipients onto a core tablet bed in a perforated coating pan.

The extended release properties of the pharmaceutical composition of the present invention may be demonstrated by monitoring the dissolution of the active ingredient. The dissolution of the active ingredient may be monitored using standard procedures well known to those skilled in the art (e.g. the dissolution test procedures, such as the Rotating Basket Method (Apparatus I) or Paddle Method (Apparatus II), disclosed in the U.S. Pharmacopeia (USP). Such procedures include those in which the formulation is immersed in an aqueous medium such as water or hydrochloric acid and aliquots of the medium are withdrawn at various time points over a period of 24 hours. The aliquots are analyzed using high pressure liquid chromatography (HPLC) with UV detection to determine the concentration of dissolved active ingredient using standard methodology.

In a particular embodiment, the dissolution profile is determined by the Paddle Method by immersing a tablet in 500 ml, Modified SGF, pH 1.0 (NaCl 0.2% w/w in 0.0825N HCl) USP II, 50 RPM.

The various embodiments of the present invention can be assembled in several different ways.

In one embodiment, the present invention provides a non-osmotic coated extended release pharmaceutical composition comprising Paliperidone or pharmaceutically acceptable salts thereof and one or more pharmaceutical excipients coated with release controlling composition wherein the release of active is solely controlled by coating.

In yet another embodiment, the present invention provides a non-osmotic coated extended release pharmaceutical composition comprising Paliperidone or pharmaceutically acceptable salts thereof and one or more pharmaceutical excipients coated with a release controlling composition comprising one or more hydrophobic agents and one or more hydrophilic agents.

In yet another embodiment, the present invention provides a non-osmotic coated extended release pharmaceutical composition comprising immediate release core comprising Paliperidone or pharmaceutically acceptable salts thereof and one or more pharmaceutical excipients wherein the core is coated with a release controlling composition comprising one or more hydrophobic agents and one or more hydrophilic agents.

In yet another embodiment the present invention provides a non-osmotic coated extended release pharmaceutical composition comprising immediate release core comprising Paliperidone or pharmaceutically acceptable salts thereof and one or more pharmaceutical excipients and wherein the core is coated with a release controlling composition comprising ethylcellulose and polyethylene glycol.

In yet another embodiment, the present invention provides a process of preparing coated extended release pharmaceutical composition comprising immediate release core comprising Paliperidone or pharmaceutically acceptable salts thereof and one or more pharmaceutical excipients; coated with a release controlling composition comprising one or more hydrophobic agents and one or more hydrophilic agents.

In yet another embodiment, the present invention provides a non-osmotic coated extended release tablet comprising a core comprising Paliperidone or pharmaceutically acceptable salts thereof and one or more pharmaceutical excipients wherein the core is coated with a release controlling composition comprising one or more hydrophobic agents and one or more hydrophilic agents.

In yet another embodiment, the present invention provides a non-osmotic coated extended release tablet comprising a immediate release core comprising Paliperidone or pharmaceutically acceptable salts thereof and one or more pharmaceutical excipients wherein the core is coated with a release controlling composition comprising one or more hydrophobic agents and one or more hydrophilic agents.

In yet another embodiment, the present invention provides a non-osmotic coated extended release tablet comprising a core comprising Paliperidone or pharmaceutically acceptable salts thereof and one or more pharmaceutical excipients wherein the core is coated with a release controlling composition comprising ethylcellulose and polyethylene glycol.

In yet another embodiment, the present invention provides a non-osmotic coated extended release tablet comprising immediate release core comprising Paliperidone or pharmaceutically acceptable salts thereof and one or more pharmaceutical excipients wherein the core is coated with a release controlling composition comprising ethylcellulose and polyethylene glycol.

In yet another embodiment, the present invention provides a non-osmotic coated extended release tablet comprising immediate release core comprising Paliperidone or pharmaceutically acceptable salts thereof and one or more pharmaceutical excipients wherein the core is coated with a release controlling composition comprising cellulose acetate and polyethylene glycol.

In yet another embodiment, the present invention provides a process of preparing coated extended release pharmaceutical composition comprising immediate release core comprising Paliperidone or pharmaceutically acceptable salts thereof and one or more pharmaceutical excipients wherein the core is coated with a release controlling composition comprising one or more hydrophobic agents and one or more hydrophilic agents wherein process can be selected from direct compression, dry granulation, wet granulation (aqueous/non-aqueous or combination) and melt granulation.

The following examples illustrate preferred embodiments in accordance with the present invention without limiting the scope or spirit of the invention.

### EXAMPLE NO. 1

| | |
|---|---|
| | Composition |
| | A |
| Ingredients | Quantity mg/Tablet |

Core Tablet Composition

| | |
|---|---|
| Paliperidone | 3.0 |
| Lactose Monohydrate | 94.0 |
| Povidone | 2.0 |
| Purified water | q.s. |
| Magnesium Stearate | 1.0 |
| Total | 100.0 |

Release Controlling Coating Composition

| | |
|---|---|
| Ethylcellulose (Ethocel Standard 7 Premium) | 10.0 |
| PEG 6000 | 7.5 |
| Dichloromethane | q.s. |
| Total | 117.5 |

Manufacturing Procedure

Sift paliperidone, lactose monohydrate through #40 mesh sieve and mix geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed Magnesium Stearate. Compress the lubricated blend into tablets by using suitable punches.

Preparation the Coating Solution:

Dissolve Ethylcellulose and PEG 6000 in sufficient quantity of dichloromethane. Film coat the tablets by using coating solution to a weight gain of 2 to 30% w/w.

### EXAMPLE NO. 2

| | |
|---|---|
| | Composition |
| | B |
| Ingredients | Quantity mg/Tablet |

Core Tablet Composition

| | |
|---|---|
| Paliperidone | 6.0 |
| Lactose Monohydrate | 188.0 |
| Povidone | 4.0 |
| Purified water | q.s. |
| Magnesium Stearate | 2.0 |
| Total | 200.0 |

Release Controlling Coating Composition

| | |
|---|---|
| Ethylcellulose (Ethocel Standard 45 Premium) | 10.0 |
| PEG 6000 | 5.0 |
| Dichloromethane | q.s. |
| Total | 215.0 |

Manufacturing Procedure

Sift paliperidone, lactose monohydrate through #40 mesh sieve and mix geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed magnesium stearate. Compress the lubricated blend into tablets by using suitable punches.

Preparation of the Coating Solution:

Dissolve Ethylcellulose and PEG 6000 in sufficient quantity of dichloromethane. Film coat the tablets by using coating solution to a weight gain of 2 to 30% w/w.

### EXAMPLE NO. 3

| Composition | | |
|---|---|---|
| C | D | E |

| | |
|---|---|
| Ingredients | Quantity mg/Tablet |

Core Tablet Composition

| | | | |
|---|---|---|---|
| Paliperidone | 6.0 | 6.0 | 6.0 |
| Lactose Monohydrate | 188.0 | 188.0 | 188.0 |
| Povidone | 4.0 | 4.0 | 4.0 |
| Purified water | q.s. | q.s. | q.s. |
| Magnesium Stearate | 2.0 | 2.0 | 2.0 |
| Total | 200.0 | 200.0 | 200.0 |

Release Controlling Coating Composition

| | | | |
|---|---|---|---|
| Ethylcellulose (Ethocel Standard 100 Premium) | 10.0 | 8.0 | 10.0 |
| Isopropyl Alcohol | q.s. | q.s. | q.s. |
| Ethanol | q.s. | q.s. | q.s. |
| PEG 6000 | 5.0 | 5.0 | 7.5 |
| Purified Water | q.s. | q.s. | q.s. |
| Total | 215.0 | 213.0 | 217.5 |

Manufacturing Procedure

Sift paliperidone, lactose monohydrate through #40 mesh sieve and mix geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed Magnesium Stearate. Compress the lubricated blend into tablets by using suitable punches.

Preparation of Coating Solution:

a. Dissolve Ethylcellulose in mixture of Isopropyl alcohol:Ethanol in 1:1 ratio.

b. Dissolve PEG 6000 in sufficient quantity of purified water.

c. Add solution of Step (b) in solution of Step (a) and mix to get clear solution

Film coat the tablet by using the coating solution to a weight gain of 2 to 30% w/w.

### EXAMPLE NO. 4

| | |
|---|---|
| | Composition |
| | F |
| Ingredients | Quantity mg/Tablet |

Core Tablet Composition

| | |
|---|---|
| Paliperidone | 6.0 |
| Lactose Monohydrate | 188.0 |
| Povidone | 4.0 |
| Purified water | q.s. |
| Magnesium Stearate | 2.0 |
| Total | 200.0 |

Under Coating composition

| | |
|---|---|
| Ethyl Cellulose (Ethocel Standard 100 Premium) | 10.0 |
| Isopropyl Alcohol | q.s. |
| Ethanol | q.s. |
| PEG 6000 | 7.5 |
| Purified Water | q.s. |
| Total | 217.5 |

Over coating composition

| | |
|---|---|
| Hypromellose (Methocel K4M Premium) | 11.0 |
| Polyethylene Glycol | 3.0 |
| Ethanol | q.s. |
| Purified Water | q.s. |
| Total | 231.5 |

Manufacturing Procedure

Sift paliperidone, lactose monohydrate through #40 mesh sieve and mix geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed Magnesium Stearate. Compress the lubricated blend into tablets by using suitable punches.

Preparation of Under Coating Solution:

a. Dissolve Ethylcellulose in mixture of Isopropyl alcohol:Ethanol in 1:1 ratio.

b. Dissolve PEG 6000 in sufficient quantity of purified water.

c. Add solution of Step (b) in solution of Step (a) and mix to get clear solution

Film coat the tablet by using the coating solution to a weight gain of 2 to 30% w/w.

Preparation of Over Coating Solution:

a) Dissolve Hypromellose in sufficient quantity of ethanol.

b) Dissolve PEG 6000 in sufficient quantity of purified water.

c) Add Step b) solution into step a( ) solution and mix.

Film coat the tablets coated with undercoat coating solution by over coating solution to a weight gain of 5 to 30% w/w.

### EXAMPLE NO. 5

| | |
|---|---|
| | Composition |
| | G |
| Ingredients | Quantity mg/Tablet |

Core Tablet Composition

| | |
|---|---|
| Paliperidone | 6.0 |
| Lactose Monohydrate | 188.0 |
| Povidone | 4.0 |
| Purified water | q.s. |
| Magnesium Stearate | 2.0 |
| Total | 200.0 |

Release Controlling Coating Composition

| | |
|---|---|
| Cellulose Acetate (CA 398-10) | 20.0 |
| Sorbitol | 6.0 |
| PEG 400 | 3.0 |
| Acetone | q.s |
| Purified Water | q.s. |
| Total | 229.0 |

Manufacturing Procedure

Sift paliperidone, lactose monohydrate through #40 mesh sieve and mix geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed magnesium stearate. Compress the lubricated blend into tablets by using suitable punches.

Preparation of Coating Solution:

a. Dissolve Cellulose Acetate in sufficient quantity of acetone.

b. Dissolve Sorbitol and PEG 400 in sufficient quantity of purified water.

c. Add solution of Step (b) in solution of Step (a) and mix to get clear solution

Film coat the tablets by using coating solution to a weight gain of 2 to 30% w/w.

### EXAMPLE NO. 6

| Composition | |
|---|---|
| H | I |

| | |
|---|---|
| Ingredients | Quantity mg/Tablet |

Core Tablet Composition

| | |
|---|---|
| Paliperidone | 6.0 |
| Hydroxy Propyl Betacyclodextrin (Kleptose HPB) | 6.0 |
| Lactose Monohydrate | 182.0 |
| Povidone | 4.0 |
| Purified water | q.s. |
| Magnesium Stearate | 2.0 |
| Total | 200.0 |

Release Controlling Coating Composition

| | | |
|---|---|---|
| Cellulose Acetate (CA 398-10) | 10.0 | 15.79 |
| Sorbitol | 6.0 | 9.47 |
| PEG 400 | 3.0 | 4.74 |
| Acetone | q.s | q.s |
| Purified Water | q.s. | q.s. |
| Total | 219.0 | 230.0 |

Manufacturing Procedure

Sift paliperidone, lactose monohydrate and Hydroxy Propyl Betacyclodextrin through #40 mesh sieve. Mix Paliperidone with Hydroxy Propyl Betacyclodextrin and then with lactose monohydrate geometrically Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed magnesium stearate. Compress the lubricated blend into tablets by using suitable punches.

Preparation of Coating Solution:

a. Dissolve Cellulose Acetate in sufficient quantity of acetone.

b. Dissolve Sorbitol and PEG 400 in sufficient quantity of purified water.

c. Add solution of Step (b) in solution of Step (a) and mix to get clear solution

Film coat the tablets by using coating solution to a weight gain of 2 to 30% w/w.

| | |
|---|---|
| | Composition |
| | J |
| Ingredients | Quantity mg/Tablet |

Core Tablet Composition

| | |
|---|---|
| Paliperidone | 6.0 |
| Hydroxy Propyl Betacyclodextrin (Kleptose HPB) | 6.0 |
| Lactose Monohydrate | 182.0 |
| Povidone | 4.0 |
| Purified water | q.s. |
| Magnesium Stearate | 2.0 |
| Total | 200.0 |

Release Controlling Coating Composition

| | |
|---|---|
| Ethylcellulose | 8.0 |
| PEG 6000 | 5.0 |
| Ethanol | q.s. |
| Isopropyl Alcohol | q.s |
| Purified Water | q.s. |
| Total | 213.0 |

### EXAMPLE NO. 7

Manufacturing Procedure

Sift paliperidone, lactose monohydrate and Hydroxy Propyl Betacyclodextrin through #40 mesh sieve. Mix Paliperidone with Hydroxy Propyl Betacyclodextrin and then with lactose monohydrate geometrically. Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed magnesium stearate. Compress the lubricated blend into tablets by using suitable punches.

Preparation of Coating Solution:

a. Dissolve Ethylcellulose in mixture of Isopropyl alcohol:Ethanol in 1:1 ratio.

b. Dissolve PEG 6000 in sufficient quantity of purified water.

c. Add solution of Step (b) in solution of Step (a) and mix to get clear solution

Film coat the tablets by using coating solution to a weight gain of 2 to 30% w/w.

### EXAMPLE NO. 8

| Composition | |
|---|---|
| K | L |

| | |
|---|---|
| Ingredients | Quantity mg/Tablet |

Core Tablet Composition

| | | |
|---|---|---|
| Paliperidone | 6.0 | 6.0 |
| Hydroxy Propyl Betacyclodextrin (Kleptose HPB) | 6.0 | — |
| Lactose Monohydrate | 182.0 | 188.0 |
| Povidone | 4.0 | 4.0 |
| Purified water | q.s. | q.s. |
| Magnesium Stearate | 2.0 | 2.0 |
| Total | 200.0 | 200.0 |

Release Controlling Coating Composition

| | | |
|---|---|---|
| Cellulose Acetate (CA 398-10) | 19.44 | 26.31 |
| Sorbitol | 11.67 | 15.79 |
| PEG 400 | 3.89 | 7.90 |
| Acetone | q.s | q.s |
| Purified Water | q.s. | q.s. |
| Total | 235.0 | 250.0 |

Manufacturing Procedure

Sift paliperidone, lactose monohydrate and Hydroxy Propyl Betacyclodextrin through #40 mesh sieve. Mix Paliperidone with Hydroxy Propyl Betacyclodextrin and then with lactose monohydrate geometrically Sift the blend through #40 mesh sieve and mix again. Granulate the blend by using aqueous povidone solution. Dry the granules at temperature NMT 60° C. to get the LOD<2.0% w/w. Pass the dried granules through #30 mesh sieve and lubricate with #40 mesh sieve passed magnesium stearate. Compress the lubricated blend into tablets by using suitable punches.

Preparation of Coating Solution:

a. Dissolve Cellulose Acetate in sufficient quantity of acetone.

b. Dissolve Sorbitol and PEG 400 in sufficient quantity of purified water.

c. Add solution of Step (b) in solution of Step (a) and mix to get clear solution

Film coat the tablets by using coating solution to a weight gain of 2 to 30% w/w.

### EXAMPLE NO. 9

| | |
|---|---|
| | Composition |
| | M |
| Ingredients | Quantity mg/Tablet |

Core Tablet Composition

| | |
|---|---|
| Paliperidone | 6.0 |
| Lactose Monohydrate | 186.0 |
| Povidone | 6.0 |
| Purified water | q.s. |
| Magnesium Stearate | 2.0 |
| Total | 200.0 |

Release Controlling Coating Composition

| | |
|---|---|
| Cellulose Acetate (CA 398-10) | 16.7 |
| Opadry Clear (03K19229) | 3.3 |
| Acetone | q.s |
| Purified Water | q.s. |
| Total | 220.0 |

Manufacturing Procedure

Sift paliperidone, lactose monohydrate through #40 mesh sieve. Mix Paliperidone with lactose monohydrate geometrically and sift the blend through #40 mesh. Mix the blend in rapid mixer granulator and granulate with aqueous povidone solution. Dry the granules and pass the dried granules through #30 mesh sieve. Lubricate the granules with #40 mesh sieve passed magnesium stearate in blender. Compress the lubricated blend into tablets by using suitable punches.

Preparation of Coating Solution:

a. Dissolve Cellulose Acetate in sufficient quantity of acetone.

b. Dissolve Opadry Clear (03K19229) in sufficient quantity of purified water.

c. Add solution of Step (b) in solution of Step (a) and mix to get clear solution

Coat the tablets by using coating solution to a weight gain of 2 to 30% w/w.

### EXAMPLE NO. 10

| | |
|---|---|
| | Composition |
| | N |
| Ingredients | Quantity mg/Tablet |

Core Tablet Composition

| | |
|---|---|
| Paliperidone | 6.0 |
| Lactose Monohydrate | 186.0 |
| Povidone | 6.0 |
| Purified water | q.s. |
| Magnesium Stearate | 2.0 |
| Total | 200.0 |

Release Controlling Coating Composition

| | |
|---|---|
| Cellulose Acetate (CA 398-10) | 6.15 |
| Hydroxypropylmethylcellulose | 9.23 |
| Polyethylene Glycol | 0.62 |
| Acetone | q.s |
| Methylene Chloride | q.s. |
| Total | 216.0 |

Manufacturing Procedure

Sift paliperidone, lactose monohydrate through #40 mesh sieve. Mix Paliperidone with lactose monohydrate geometrically and sift the blend through #40 mesh. Mix the blend in rapid mixer granulator and granulate with aqueous povidone solution. Dry the granules and pass the dried granules through #30 mesh sieve. Lubricate the granules with #40 mesh sieve passed magnesium stearate in blender. Compress the lubricated blend into tablets by using suitable punches.

Preparation of Coating Solution:

a. Dissolve Cellulose Acetate in sufficient quantity of acetone.

b. Dissolve HPMC and PEG in sufficient quantity of methylene chloride.

c. Add solution of Step (b) in solution of Step (a) and mix to get clear solution

Coat the tablets by using coating solution to a weight gain of 2 to 30% w/w.

The formulations of Example No. 1 to Example No. 10 were subjected to in-vitro dissolution studies and the results obtained in comparison with INVEGA® 3 mg, 6 mg and the results obtained are presented below table:

| | Composition | | | | | | | | | | | | | | | |
|---|---|---|---|---|---|---|---|---|---|---|---|---|---|---|---|---|
| | INVEGA ® | INVEGA ® | | | | | | | | | | | | | | |
| | 3 mg | 6 mg | A | B | C | D | E | F | G | H | I | J | K | L | M | N |
| | Dissolution Condition | | | | | | | | | | | | | | | |
| | 500 ml, Modified SGF, pH 1.0 (NaCl 0.2% w/w in 0.0825N HCl) USP II, 50 RPM | | | | | | | | | | | | | | | |
| Time | Cumulative % Drug Released | | | | | | | | | | | | | | | |
| 1 | 0 | 1 | 32 | 6 | 0 | 1 | 5 | 0 | 0 | — | 4 | 1 | 0 | 1 | 0 | 0 |
| 2 | 1 | — | 59 | 16 | — | — | — | — | — | — | — | — | — | 7 | 0 | 0 |
| 4 | 4 | 5 | 86 | 34 | 4 | 8 | 30 | 5 | 2 | 39 | 14 | 10 | 4 | 17 | 0 | 0 |
| 6 | 8 | 13 | 97 | 50 | 7 | — | — | — | — | — | — | 19 | 10 | — | 2 | 1 |
| 8 | 17 | 22 | 108 | 59 | 11 | 21 | 57 | 12 | 4 | 69 | 25 | 27 | 16 | 35 | 4 | 21 |
| 12 | 42 | 45 | — | 89 | 20 | 33 | 79 | 22 | 6 | 90 | 42 | 42 | 28 | 52 | 33 | 54 |
| 14 | 52 | 59 | — | 99 | 25 | — | — | — | — | 99 | — | 51 | 35 | 61 | 50 | 68 |
| 18 | 74 | 86 | — | 105 | 36 | 59 | 99 | 34 | 9 | 106 | 83 | 66 | 48 | 81 | 74 | 86 |
| 20 | 90 | 102 | — | — | 40 | 65 | 102 | 46 | 12 | — | 98 | 72 | 52 | 89 | 81 | 92 |
| 24 | 102 | 108 | — | — | 57 | 76 | 108 | 59 | 16 | — | 109 | 82 | 69 | 104 | 90 | 99 |

We claim:

1. A non-osmotic coated extended release pharmaceutical composition comprising immediate release core comprising Paliperidone or pharmaceutically acceptable salts thereof and one or more pharmaceutical excipients wherein the core is coated with a release controlling composition comprising one or more hydrophobic agents and one or more hydrophilic agents.

2. A non-osmotic coated extended release pharmaceutical composition according to claim 1 wherein the ratio of the hydrophobic agent to the hydrophilic agent is from about 0.1:10 to about 10:0.1.

3. A non-osmotic coated extended release pharmaceutical composition according to claim 1 wherein Paliperidone or pharmaceutically acceptable salts thereof is present from about 0.1% w/w to about 25% w/w of composition.

4. A non-osmotic coated extended release pharmaceutical composition according to claim 1 wherein hydrophobic agents can be selected from group consisting of polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), Poly(methyl acrylate-co-methyl methacrylate-co-methacrylic acid), Poly(methacrylic acid-co-ethyl acrylate), Poly(methacrylic acid-co-methyl methacrylate), Eudragit FS 30D, Eudragit L 100-55, Eudragit L 30D-55, EUDRAGIT® L 100, EUDRAGIT® L 12,5, EUDRAGIT® S 100, EUDRAGIT® S 12,5 Acryl Eze, beeswax, carnauba wax, paraffin wax, microcrystalline wax, ozokerite, cetostearyl alcohol, stearyl alcohol, cetyl alcohol and myristyl alcohol, and fatty acid esters such as glyceryl monostearate, glycerol monooleate, acetylated monoglycerides, tristearin, tripalmitin, cetyl esters wax, glyceryl palmitostearate, glyceryl behenate, hydrogenated vegetable oils and combinations thereof

5. A non-osmotic coated extended release pharmaceutical composition according to claim 1 wherein hydrophilic agents can be selected from group consisting of hydroxyethyl cellulose, hydroxypropyl cellulose, carboxymethyl cellulose, hydroxypropylmethyl cellulose, sodium carboxymethyl cellulose, Plasdone® S-630, polyvinyl alcohol, polyethylene glycol and combinations thereof.

6. A non-osmotic coated extended release pharmaceutical composition according to claim 1 wherein formulation is prepared using direct compression, dry granulation, wet granulation (aqueous/non-aqueous or combination) or melt granulation.

7. A non-osmotic coated extended release tablet comprising a immediate release core comprising Paliperidone or pharmaceutically acceptable salts thereof and one or more pharmaceutical excipients wherein the core is coated with a release controlling composition comprising one or more hydrophobic agents and one or more hydrophilic agents.

8. A non-osmotic coated extended release tablet according to claim 7 wherein the ratio of the hydrophobic agent to the hydrophilic agent is from about 0.1:10 to about 10:0.1.

9. A non-osmotic coated extended release tablet according to claim 7 wherein Paliperidone or pharmaceutically acceptable salts thereof is present from about 0.1% w/w to about 25% w/w of composition.

10. A non-osmotic coated extended release tablet according to claim 7 wherein hydrophobic agents can be selected from group consisting of polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), Poly(methyl acrylate-co-methyl methacrylate-co-methacrylic acid), Poly(methacrylic acid-co-ethyl acrylate), Poly(methacrylic acid-co-methyl methacrylate), Eudragit FS 30D, Eudragit L 100-55, Eudragit L 30D-55, EUDRAGIT® L 100, EUDRAGIT® L 12,5, EUDRAGIT® S 100, EUDRAGIT® S 12,5 Acryl Eze, beeswax, carnauba wax, paraffin wax, microcrystalline wax, ozokerite, cetostearyl alcohol, stearyl alcohol, cetyl alcohol and myristyl alcohol, and fatty acid esters such as glyceryl monostearate, glycerol monooleate, acetylated monoglycerides, tristearin, cetyl esters wax, glyceryl palmitostearate, glyceryl behenate, hydrogenated vegetable oils and combinations thereof.

11. A non-osmotic coated extended release tablet according to claim 7 wherein hydrophilic agents can be selected from group consisting of hydroxyethyl cellulose, hydroxypropyl cellulose, carboxymethyl cellulose, hydroxypropylmethyl cellulose, sodium carboxymethyl cellulose, Plasdone® S-630, polyvinyl alcohol, polyethylene glycol and combinations thereof.

12. A non-osmotic coated extended release tablet according to claim 7 wherein formulation is prepared using direct compression, dry granulation, wet granulation (aqueous/non-aqueous or combination) or melt granulation.

13. A non-osmotic coated extended release tablet comprising immediate release core comprising Paliperidone or pharmaceutically acceptable salts thereof and one or more pharmaceutical excipients wherein the core is coated with a release controlling composition comprising ethylcellulose and polyethylene glycol.

14. A non-osmotic coated extended release tablet according to claim 13 wherein the ratio of ethylcellulose and polyethylene glycol is from about 0.1:10 to about 10:0.1.

15. A non-osmotic coated extended release tablet according to claim 13 wherein Paliperidone or pharmaceutically acceptable salts thereof is present from about 0.1% w/w to about 25% w/w of composition.

16. A non-osmotic coated extended release tablet according to claim 13 wherein formulation is prepared using direct compression, dry granulation, wet granulation (aqueous/non-aqueous or combination) or melt granulation.

17. A non-osmotic coated extended release tablet comprising immediate release core comprising Paliperidone or pharmaceutically acceptable salts thereof and one or more pharmaceutical excipients wherein the core is coated with a release controlling composition comprising cellulose acetate and polyethylene glycol.

18. A non-osmotic coated extended release tablet according to claim 17 wherein the ratio of cellulose acetate and polyethylene glycol is from about 0.1:10 to about 10:0.1.

19. A non-osmotic coated extended release tablet according to claim 17 wherein Paliperidone or pharmaceutically acceptable salts thereof is present from about 0.1% w/w to about 25% w/w of composition.

20. A non-osmotic coated extended release tablet according to claim 17 wherein formulation is prepared using direct compression, dry granulation, wet granulation (aqueous/non-aqueous or combination) or melt granulation.
